(19) United States
(12) Patent Application Publication
(10) Pub. No.: US 2026/0199363 A1
(43) Pub. Date: Jul. 16, 2026

(54) METHOD OF TREATING CANCER IN A PATIENT WITH HEPATIC DYSFUNCTION

(71) Applicant: Servier Pharmaceuticals LLC, Boston, MA (US)

(72) Inventors: Shuchi Pandya, Newton, MA (US); Tharin Limsakun, Union, NJ (US); Mohammad Hossain, Exton, PA (US)

(21) Appl. No.: 19/018,659

(22) Filed: Jan. 13, 2025

Related U.S. Application Data
(60) Provisional application No. 63/612,322, filed on Dec. 19, 2023.

(51) Int. Cl. A61K 31/53; A61K 9/20; A61P 35/00

(52) U.S. Cl. CPC A61K 31/53; A61K 9/2086; A61P 35/00

(57) ABSTRACT

Provided are methods for treating brain tumors, characterized by an IDH1 and/or IDH2 mutation, with vorasidenib, in patients with hepatic impairment.

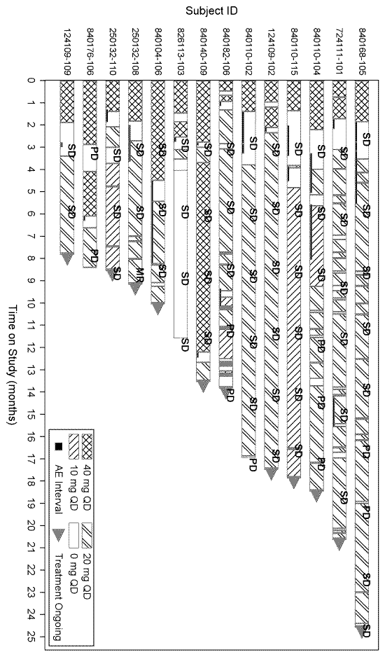

### FIELD

The present invention relates to methods for treating brain tumors with vorasidenib, in patients with hepatic impairment.

### BACKGROUND

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 (peroxisomal targeting signal-1) sequence. The presence of this enzyme in peroxisomes suggests roles in the regeneration of NADPH for intraperoxisomal reductions, such as the conversion of 2, 4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (DEC-2008) to UniProtKB; Kullmann et al., Submitted (JUN-1996) to the EMBL/GenBank/DDBJ databases; and Sjoeblom et al., Science 314:268-274(2006).

Non-mutant, e.g., wild type, IDH1 catalyzes the oxidative decarboxylation of isocitrate to α-ketoglutarate thereby reducing NAD+ (NADP+) to NADH (NADPH), e.g., in the forward reaction:

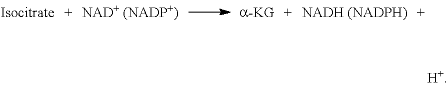

It has been discovered that mutations of IDH1 present in certain cancer cells result in a new ability of the enzyme to catalyze the NADPH-dependent reduction of α-ketoglutarate to R(−)-2-hydroxyglutarate (2HG). The production of 2HG is believed to contribute to the formation and progression of cancer (Dang, L et al, Nature 2009, 462:739-44).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_002168.2 and NP_002159.2 respectively. The nucleotide and amino acid sequence for human IDH2 are also described in, e.g., Huh et al., Submitted (NOV-1992) to the EMBL-GenBank/DDBJ databases; and the MGC Project team, Genome Res. 14:2121-2127(2004).

Non-mutant, e.g., wild type, IDH2 catalyzes the oxidative decarboxylation of isocitrate to a-ketoglutarate (a-KG).

It has been discovered that mutations of IDH2 present in certain cancer cells result in a new ability of the enzyme to catalyze the NADPH-dependent reduction of α-ketoglutarate to R(−)-2-hydroxyglutarate (2HG). 2HG is not formed by wild-type IDH2. The production of 2HG is believed to contribute to the formation and progression of cancer (Dang, L et al, Nature 2009, 462:739-44). The inhibition of mutant IDH1 and/or mutant IDH2 and their neomorphic activity is therefore a potential therapeutic treatment for cancer.

Oligodendrogliomas and astrocytomas are primary brain tumors and represent subtypes of gliomas (e.g., adult-type diffuse gliomas). Traditionally, oligodendrogliomas and astrocytomas have been classified in accordance with their histopathological phenotypes, but more recent classification guidelines have moved toward an increased role of molecular markers in CNS tumor taxonomy. Per 2021 WHO (World Health Organization) classification of CNS tumors (WHO CNS5), adult-type diffuse gliomas are classified into three subtypes: oligodendroglioma, astrocytoma and glioblastoma. Oligodendrogliomas harbor IDH1 or IDH2 mutations and 1p19q co-deletion. Per CNS5, oligodendrogliomas occur as grade 2 or 3 tumors. Astrocytomas harbor IDH1 or IDH2 mutations but lack 1p19q co-deletion. Per CNS5, astrocytomas occur as grade 2, 3 or 4 tumors. Finally, glioblastomas are characterized by a lack of IDH mutations (i.e., wild-type IDH). (Louis, D. N. et al., Neuro-Oncology, 23: 1231-1251(2021)).

Recurrent or progressive IDH-mutant gliomas are oligodendrogliomas and astrocytomas that harbor IDH1 or IDH2 gene mutation and have recurred or progressed after receiving standard of care therapy including surgery, radiation and/or chemotherapy.

Vorasidenib is an orally available, brain penetrant second-generation dual mutant isocitrate dehydrogenase 1 and 2 (mIDH1/2) inhibitor currently being evaluated in clinical trials for the treatment of IDH-mutant gliomas.

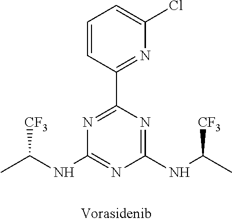

Vorasidenib or (6-(6-chloropyridin-2-yl)-N²,N⁴-bis((R)-1,1,1-trifluoropropan-2-yl)-1,3,5-triazine-2,4-diamine) is disclosed in U.S. Pat. No. 9,579,324, which is incorporated herein by reference in its entirety.

Vorasidenib is predominantly excreted via feces. So, it is theoretically considered that, when vorasidenib is administered to patient with hepatic impairment, exposure to vorasidenib can be increased. Further, alanine transferase (ALT) and aspartate transferase (AST) elevations were observed in clinical trials with vorasidenib, suggesting that vorasidenib affects hepatic function; elevated liver transaminases were designated as adverse events of special interest in the AG881-C-004 (NCT04164901) clinical trial. Safe and effective dosing in patients with hepatic impairment is a challenge, and selection of the correct dosage regiment is typically based on pharmacokinetics (PK) according to FDA guidance, and generally requires clinical trials. The influence of hepatic impairment on the PK of vorasidenib was evaluated in a dedicated clinical pharmacology study. However, it may not be easy to perform cancer treatment that is safe and has effectiveness on a cancer patient with severe hepatic impairment.

### SUMMARY

The present invention relates to provision of a method for treating a brain tumor with vorasidenib in a patient with hepatic impairment.

In one embodiment, provided is vorasidenib for use in a method of treating a brain tumor in a patient with hepatic impairment.

In one embodiment, provided herein is a pharmaceutical composition comprising 10 mg of vorasidenib or 40 mg of vorasidenib for use in a method for treating a brain tumor, in a patient with hepatic impairment.

The present invention relates to provision of a method for treating a brain tumor with vorasidenib in a patient that has developed elevated liver transaminases upon administration of single or multiple doses of vorasidenib 40 mg QD.

In one embodiment, provided is vorasidenib for use in a method of treating a brain tumor in a patient in a patient that has developed elevated liver transaminases upon administration of single or multiple doses of vorasidenib 40 mg QD.

In one embodiment, provided herein is a pharmaceutical composition comprising 10 mg of vorasidenib or 40 mg of vorasidenib for use in a method for treating a brain tumor, in a patient that has developed elevated liver transaminases upon administration of single or multiple doses of vorasidenib 40 mg QD.

The present invention relates to provision of a method for treating a brain tumor with vorasidenib in a patient with mild or moderate hepatic impairment comprising administering to the patient a dose of 40 mg vorasidenib QD.

In one embodiment, provided is vorasidenib for use in a method of treating a brain tumor in a patient in a patient in a patient with mild or moderate hepatic impairment by administering to the patient 40 mg vorasidenib QD.

In one embodiment, provided herein is a pharmaceutical composition comprising 10 mg of vorasidenib or 40 mg of vorasidenib for use in a method for treating a brain tumor in a patient with mild or moderate hepatic impairment wherein the method comprises administering to the patient 40 mg vorasidenib QD.

The present invention especially relates to provision of a method of treatment of a brain tumor, characterized by an IDH1 and/or IDH2 mutation, in a patient treated with vorasidenib 40 mg/day, once a day (QD), and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   At 40 mg/day, once a day, if ALT or AST are more than 3-fold but less than or equal to 5 fold of ULN, at first occurrence of the elevation,
   At 20 mg/day, once a day, if ALT or AST are more than 3-fold but less than or equal to 5 fold of ULN, at second occurrence of the elevation,
   At 10 mg/day, once a day, if ALT or AST are more than 3-fold but less than or equal to 5 fold of ULN, at third occurrence of the elevation,
   At 20 mg/day, once a day, if ALT or AST are more than 5-fold but less than or equal to 20 fold of ULN, at first occurrence of the elevation.

The present invention also relates to a method of treatment of a brain tumor, characterized by the presence of a mutant allele of IDH1 and/or IDH2, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubin superior or equal to 2-fold of ULN comprising weekly monitoring Liver Function Tests until resolution.

The present invention relates to vorasidenib for use in a method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   At 40 mg/day, once a day, if ALT or AST are more than 3-fold but less than or equal to 5 fold of ULN, at first occurrence of the elevation,
   At 20 mg/day, once a day, if ALT or AST are more than 3-fold but less than or equal to 5 fold of ULN, at second occurrence of the elevation,
   At 10 mg/day, once a day, if ALT or AST are more than 3-fold but less than or equal to 5 fold of ULN, at third occurrence of the elevation,
   At 20 mg/day, once a day, if ALT or AST are more than 5-fold but less than or equal to 20 fold of ULN, at first occurrence of the elevation.

The present invention also relates to vorasidenib for use in a method of treatment of a brain tumor, characterized by the presence of a mutant allele of IDH1 and/or IDH2, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubin superior or equal to 2-fold of ULN comprising weekly monitoring Liver Function Tests until resolution.

The present invention relates to pharmaceutical composition comprising 10 mg of vorasidenib or 40 mg of vorasidenib for use in a method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   At 40 mg/day, once a day, if ALT or AST are more than 3-fold but less than or equal to 5 fold of ULN, at first occurrence of the elevation,
   At 20 mg/day, once a day, if ALT or AST are more than 3-fold but less than or equal to 5 fold of ULN, at second occurrence of the elevation,
   At 10 mg/day, once a day, if ALT or AST are more than 3-fold but less than or equal to 5 fold of ULN, at third occurrence of the elevation,
   At 20 mg/day, once a day, if ALT or AST are more than 5-fold but less than or equal to 20 fold of ULN, at first occurrence of the elevation.

The present invention relates to a pharmaceutical composition comprising 10 mg of vorasidenib or 40 mg of vorasidenib for use in a method for treating a brain tumor, characterized by the presence of a mutant allele of IDH1 and/or IDH2, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubin superior or equal to 2-fold of ULN comprising weekly monitoring Liver Function Tests until resolution.

The present invention especially relates to provision of a method of treatment of a brain tumor, characterized by an IDH1 and/or IDH2 mutation, in a patient with mild or moderate hepatic impairment, comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention relates to vorasidenib for use in a method of treatment of a brain tumor, characterized by an IDH1 and/or IDH2 mutation, in a patient with mild or moderate hepatic impairment comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention relates to pharmaceutical composition comprising 10 mg of vorasidenib or 40 mg of vorasidenib for use in a method of treatment of a brain tumor, characterized by an IDH1 and/or IDH2 mutation, in a patient with mild or moderate hepatic impairment comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

In some embodiments, the brain tumor is characterized by the presence of an IDH1 mutation, wherein the IDH1 mutation results in accumulation of R(−)-2-hydroxyglutarate in a patient.

In some embodiments, the IDH1 mutation is an R132X mutation.

In some embodiments, the IDH1 mutation is an R132H, R132C, R132S, R132G or R132L mutation.

In some embodiments, the brain tumor is characterized by the presence of an IDH2 mutation, wherein the IDH2 mutation results in accumulation of R(−)-2-hydroxyglutarate in a patient.

In some embodiments, the IDH2 mutation is an R140X mutation.

In some embodiments, the IDH2 mutation is an R140Q, R140W, or R140L mutation.

In some embodiments, the IDH2 mutation is an R172X mutation.

In some embodiments, the IDH2 mutation is an R172K or R172G mutation.

In some embodiments, the brain tumor is characterized by the presence of an IDH1 mutation and an IDH2 mutation, wherein the IDH1 and IDH2 mutations collectively result in accumulation of R(−)-2-hydroxyglutarate in a patient.

In some embodiments, the brain tumor is a glioma. In some embodiments, the brain tumor is oligodendroglioma or astrocytoma.

In some embodiments, the brain tumor is non-enhancing oligodendroglioma or astrocytoma.

In some embodiments, the brain tumor is recurrent or progressive.

In some embodiments, vorasidenib is administered in non-salt form.

In some embodiments, vorasidenib is administered as a cocrystal or a solvate thereof.

In some embodiments, vorasidenib is administered as a cocrystal with citric acid.

In some embodiments, vorasidenib is administered as an hemicitric acid hemihydrate cocrystal.

In some embodiments, vorasidenib is administered at a dose between about 10 mg/day and about 40 mg/day.

In some embodiments, vorasidenib is administered at a dose of about 10 mg/day, about 20 mg/day or about 40 mg/day.

In some embodiments, vorasidenib is administered at a daily dose between about 10 mg, and about 40 mg of vorasidenib.

In some embodiments, vorasidenib is administered at a daily dose of about 10 mg, about 20 mg or about 40 mg of vorasidenib.

In some embodiments, vorasidenib is administered once or twice daily.

In some embodiments, vorasidenib is administered once daily.

### BRIEF DESCRIPTION OF THE FIGURE

The figure shows a swimming plot of the disease response by Blinded Independent Central Review (BIRC) among 14 subjects with dose reductions due to hepatotoxicity adverse events AE=adverse event; MR=minor response; QD=daily; SD=stable disease; PD=progressive disease, CR=complete response, PR=partial response, NE=not evaluable.

### DETAILED DESCRIPTION

The details of construction and the arrangement of components set forth in the following description or illustrated in the drawings are not meant to be limiting. Other embodiments and different ways to practice the invention are expressly included. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Definitions

The terms “non-enhancing brain tumor” (e.g., non-enhancing glioma (e.g., non-enhancing astrocytoma, non-enhancing oligodendroglioma)) and “non contrast-enhancing brain tumor” (e.g., non contrast-enhancing glioma (e.g., non contrast-enhancing astrocytoma, non contrast-enhancing oligodendroglioma)) are used interchangeably herein to refer to brain tumors that don't show uptake of intravenous contrast agent in imaging studies (e.g., computerized tomography (CT) and magnetic resonance imaging (MRI)). Measurable non-enhancing brain tumor (e.g., measurable non-enhancing glioma (e.g., measurable non-enhancing astrocytoma, measurable non-enhancing oligodendroglioma)) are present in patients who have MRI-evaluable, measurable, non-enhancing disease, assessed at screening on 2D T2-weighted or 2D T2-weighted fluid-attenuated inversion recovery MRI with ≤4 mm slice thickness and no interslice gap. Measurable non-enhancing tumor is defined as a least 1 target lesion measuring ≥1 cm×≥1 cm (bidimensional). Enhancement that is confirmed to be minimal, non-nodular, and non-measurable and that has not changed between the 2 most recent scans (including screening scan) will be acceptable and corresponding tumor will be considered as non-enhancing tumor.

The term a “mutant IDH1/IDH2 inhibitor” or “inhibitor of IDH1/IDH2 mutant(s)” means a molecule e.g., a polypeptide, peptide, or small molecule (e.g., a molecule of less than 1,000 daltons), or aptomer, that binds to an IDH1/IDH2 mutant subunit and inhibits neoactivity, e.g., by inhibiting formation of a dimer, e.g., a homodimer of mutant IDH1/IDH2 subunits or a heterodimer of a mutant and a wild type subunit. In some embodiments, the neoactivity inhibition is at least about 60%, 70%, 80%, 90%, 95% or 99% as compared to the activity in the absence of the mutant IDH1/IDH2 inhibitor. In one embodiment, the mutant IDH1/IDH2 inhibitor is vorasidenib.

The term “elevated levels of 2HG” means 10%, 20% 30%, 50%, 75%, 100%, 200%, 500% or more 2HG is present in a subject that carries a mutant IDH1/IDH2 allele than is present in a subject that does not carry a mutant IDH1/IDH2 allele. The term “elevated levels of 2HG” may refer to the amount of 2HG within a cell, within a tumor, within an organ comprising a tumor, or within a bodily fluid.

The term “bodily fluid” includes one or more of amniotic fluid surrounding a fetus, aqueous humour, blood (e.g., blood plasma), serum, cerebrospinal fluid, cerumen, chyme, Cowper's fluid, female ejaculate, interstitial fluid, lymph, breast milk, mucus (e.g., nasal drainage or phlegm), pleural fluid, pus, saliva, sebum, semen, serum, sweat, tears, urine, vaginal secretion, or vomit.

The terms “inhibit” or “prevent” include both complete and partial inhibition and prevention. An inhibitor may completely or partially inhibit the intended target.

The term “subject” is intended to include human and non-human animals. Exemplary human subjects include a human patient (referred to as a patient) having a disorder, e.g., a disorder described herein or a normal subject. The term “non-human animals” of one aspect of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The term “treat” means decrease, suppress, attenuate, diminish, arrest, or stabilize the development or progression of a disease/disorder (e.g., a solid tumor, including glioma, characterized by the presence of a mutant allele of IDH1/IDH2), lessen the severity of the disease/disorder or improve the symptoms associated with the disease/disorder.

The term “solid tumor” here refers to any abnormal malignant mass of tissue that does not contain cysts or liquid areas, such as breast, lung, prostate, pancreatic, colon, bladder, brain or skin tumor. Examples of solid tumors include glioma, melanoma, chondrosarcoma, cholangiocarcinoma (including intrahepatic cholangiocarcinoma (IHCC)), prostate cancer, pancreatic dual adenocarcinoma (PDAC), colon cancer or non-small cell lung cancer (NSCLC).

An amount of a compound, including a pharmaceutically acceptable salt, cocrystal, solvate, tautomer, stereoisomer, isotopologue, prodrug or a polymorph thereof, effective to treat a disorder, or a “therapeutically effective amount” or “therapeutically effective dose” refers to an amount of the compound, including a pharmaceutically acceptable salt, solvate, tautomer, stereoisomer, isotopologue, prodrug, or a polymorph thereof, which is effective, upon single or multiple dose administration to a subject, in treating a cell, or in curing, alleviating, relieving or improving a subject with a disorder beyond that expected in the absence of such treatment.

The term “crystalline” refers to a solid having a highly regular chemical structure. In particular, a crystalline vorasidenib may be produced as one or more single crystalline forms of vorasidenib. For the purposes of this application, the terms “crystalline form”, “single crystalline form” and “polymorph” are synonymous; the terms distinguish between crystals that have different properties (e.g., different XRPD patterns and/or different DSC scan results). The term “polymorph” includes pseudo polymorphs, which are typically different solvates of a material, and thus their properties differ from one another. Thus, each distinct polymorph and pseudo polymorph of vorasidenib is considered to be a distinct single crystalline form herein.

The term “substantially crystalline” refers to forms that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to a vorasidenib that is at least 70% crystalline. In other embodiments, substantially crystalline refers to a vorasidenib that is at least 90% crystalline.

The term “isolated” refers to forms that may be at least a particular weight percent of a particular crystalline form of compound. Particular weight percentages are 90%, 91%, 92%, 93%, 94%, 95%, 96%, 97%, 98%, 99%, 99.5%, 99.9%, or any percentage between 90% and 100%.

The term “cocrystal” refers to a crystalline solid made up of two or more neutral chemical species in a defined stoichiometric ratio that possesses distinct crystallographic and spectroscopic properties when compared to the species individually. A “cocrystal” is distinct from a “salt,” which is made up of charged-balanced charged species. The species making up a cocrystal typically are linked by hydrogen bonding and other non-covalent and non-ionic interactions. Thus, a pharmaceutical cocrystal of a drug typically comprises the drug and one or more coformers. The combinations of drug and coformer(s) that will form cocrystals generally cannot be predicted ab initio, and cocrystal formation typically affects the physicochemical properties of a drug in unpredictable ways.

The term “solvate or solvated” means a physical association of a compound, including a crystalline form thereof, of this invention with one or more solvent molecules. This physical association includes hydrogen bonding. In certain instances, the solvate will be capable of isolation, for example when one or more solvent molecules are incorporated in the crystal lattice of the crystalline solid. “Solvate or solvated” encompasses both solution-phase and isolable solvates. Representative solvates include, for example, a hydrate, ethanolates or a methanolate.

The term “hydrate” is a solvate wherein the solvent molecule is H₂O that is present in a defined stoichiometric amount, and may, for example, include hemihydrate, monohydrate, dihydrate, or trihydrate.

The term “mixture” is used to refer to the combined elements of the mixture regardless of the phase-state of the combination (e.g., liquid or liquid/crystalline).

The term “pharmaceutically acceptable carrier or adjuvant” refers to a carrier or adjuvant that may be administered to a subject, together with a compound of one aspect of this invention, and which does not destroy the pharmacological activity thereof and is nontoxic when administered in doses sufficient to deliver a therapeutic amount of the compound.

The term “a pharmaceutically-acceptable salt” as used herein refers to non-toxic acid or base addition salts of the compound to which the term refers. Examples of pharmaceutically acceptable salts are discussed in Berge et al., 1977, “Pharmaceutically Acceptable Salts.” J. Pharm. Sci. Vol. 66, pp. 1-19.

The term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

### Active Compound

Vorasidenib is 6-(6-chloropyridin-2-yl)-N²,N⁴-bis((R)-1,1,1-trifluoropropan-2-yl)-1,3,5-triazine-2,4-diamine, Vorasidenib has the following chemical structure:

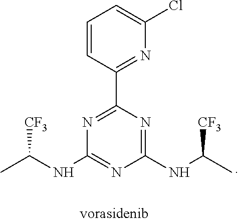

Vorasidenib is also known as AG-881, S095032 or S95032.

Vorasidenib can be prepared by the method described in paragraphs [1032]-[1036] of U.S. Publication No. 2015/0018328 A1, which paragraphs are incorporated herein by reference.

For therapeutic purposes, vorasidenib can be used in the form of a pharmaceutically acceptable salt, cocrystal, solvate, tautomer, stereoisomer, isotopologue, prodrug, or a polymorph.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” of the vorasidenib includes any non-toxic salt that, upon administration to a recipient, is capable of providing, either directly or indirectly, vorasidenib. Pharmaceutically acceptable salts are described in detail in S. M. Berge, et al., J. Pharmaceutical Sciences, 1977, 66, 1-19, incorporated herein by reference.

As used herein, the term “cocrystal” refers to a crystalline solid made up of two or more neutral chemical species in a defined stoichiometric ratio that possesses distinct crystallographic and spectroscopic properties when compared to the species individually. A “cocrystal” is distinct from a “salt,” which is made up of charged-balanced charged species. The species making up a cocrystal typically are linked by hydrogen bonding and other non-covalent and non-ionic interactions. Thus, a pharmaceutical cocrystal of a drug typically comprises the drug and one or more coformers. Cocrystals of vorasidenib (e.g., cocrystals of vorasidenib and citric acid) have been described in U.S. Publication No. 2021/0198234 A1.

As used herein, the terms “compound”, when referring to vorasidenib and cocrystals thereof, include vorasidenib and cocrystals in any form, including any tautomer or rotamer thereof, any solid form thereof (including any polymorphic form thereof), any solvate or hydrate form thereof, and any solution thereof.

In some embodiments, vorasidenib is administered as a cocrystal. In a preferred embodiment, vorasidenib is administered as a cocrystal with citric acid.

By saying that vorasidenib is administered as a cocrystal, it is meant that the compound used to prepare the pharmaceutical composition to be used in the intended method of treatment is a cocrystal of vorasidenib. Preferably, this cocrystal of vorasidenib is made with citric acid. More preferably, the compound is a hemicitric acid hemihydrate cocrystal of vorasidenib that can be represented by the formula below:

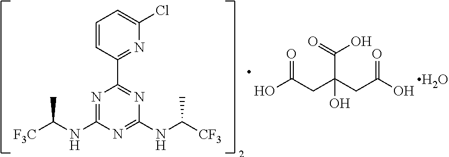

In an embodiment, vorasidenib is in the form of a hemicitric acid hemihydrate co-crystal Type A as described in WO2019090059, incorporated herein by reference in its entirety.

### Compositions and Routes of Administration

The pharmaceutical compositions used in the method of treatment of the invention may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase and combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing the compound of formula (I), or a pharmaceutically acceptable salt thereof, with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions may be administered topically to the skin. The pharmaceutical composition should be formulated with a suitable ointment containing the active components suspended or dissolved in a carrier. Carriers for topical administration of the compounds of one aspect of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of one aspect of this invention may also be topically applied to the lower intestinal tract by rectal suppository formulation or in a suitable enema formulation. Topically-transdermal patches are also included in one aspect of this invention.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In a preferred embodiment, the pharmaceutical composition is orally administered.

In one embodiment, the dosage form comprises between about 1 mg and about 100 mg vorasidenib. In one embodiment, the dosage form comprises between about 10 mg and about 40 mg vorasidenib.

In one embodiment, the dosage form comprises about 10 mg or about 40 mg of vorasidenib.

As used herein, the amount of vorasidenib comprised in the dosage form or the pharmaceutical composition refers to the amount of “free” vorasidenib. For the avoidance of doubt, a dosage form or a pharmaceutical composition comprising 10 mg of vorasidenib comprises 12.5 mg of hemicitric acid hemihydrate cocrystal of vorasidenib. Similarly, a dosage form or pharmaceutical composition comprising 40 mg of vorasidenib comprises 50.1 mg of hemicitric acid hemihydrate cocrystal of vorasidenib. Similarly, references to administration of a “dose” of vorasidenib refers to the amount of “free” vorasidenib.

### Dosage

In general, vorasidenib can be administered at a dose between about 10 mg/day and about 40 mg/day. Vorasidenib can be administered one, two, three or four times a day. In one embodiment, vorasidenib is administered one time a day (once a day, or QD). In one embodiment, vorasidenib is administered two times a day (twice daily, or BID). In one embodiment, vorasidenib is administered three times a day (TID). In one embodiment, vorasidenib is administered four times a day (QID).

In one embodiment, the dose is between about 10 mg/day and about 40 mg/day. In one embodiment, the dose is between about 10 mg/day and about 20 mg/day. In one embodiment, the dose is between about 20 mg/day and about 40 mg/day. In one embodiment, the dose is about 10 mg/day, about 20 mg/day, or about 40 mg/day.

In one embodiment, the dose is between about 10 mg and about 40 mg once daily. In one embodiment, the dose is between about 10 mg and about 20 mg once daily. In one embodiment, the dose is between about 20 mg and about 40 mg once daily. In one embodiment, the dose is about 10 mg, about 20 mg, or about 40 mg once daily.

In one embodiment, the dose is about 10 mg once daily. In one embodiment, the dose is about 20 mg once daily. In one embodiment, the dose is about 40 mg once daily.

### Methods of Treatment

The present invention relates to a method of treating a brain tumor, characterized by the presence of a mutant allele of IDH1 and/or IDH2, with vorasidenib, a cocrystal thereof or a solvate of said cocrystal in a patient with hepatic impairment. The present invention relates to a method of treating a brain tumor, characterized by the presence of a mutant allele of IDH1 and/or IDH2, with vorasidenib, a cocrystal thereof or a solvate of said cocrystal in a patient that has developed elevated liver enzymes upon administration of one or more doses of vorasidenib 40 mg QD.

In clinical trials studying the safety and efficacy of vorasidenib, in particular the INDIGO study (AG881-C-004), patients that developed elevated liver enzymes upon treatment with the standard starting dose of 40 mg vorasidenib QD were managed via a dose modification/dose interruption protocol (see Example 2). The dose modification measures were sufficient to address the elevated liver enzymes, and only a small proportion of the patients that developed elevated liver enzymes discontinued treatment due to those elevations. Additionally, an Exposure-Activity analysis investigating the effect of drug exposure on efficacy found no correlation between the levels of exposure to vorasidenib administered in accordance with protocol and any of the efficacy endpoints, indicating that dose modifications to address elevated liver enzymes do not negatively impact efficacy (Example 2).

In some embodiments, the brain tumor is a glioma. In some embodiments, the brain tumor is an oligodendroglioma or astrocytoma. In some embodiments, the brain tumor is an oligodendroglioma. In some embodiments, the brain tumor is an astrocytoma.

In some embodiments, the brain tumor is a recurrent or progressive glioma. In some embodiments, the brain tumor is a recurrent or progressive oligodendroglioma or a recurrent or progressive astrocytoma. In some embodiments, the brain tumor is a recurrent or progressive oligodendroglioma. In some embodiments, the brain tumor is a recurrent or progressive astrocytoma. In some embodiments, the brain tumor is a predominantly non-enhancing astrocytoma or oligodendroglioma. In some embodiments, the brain tumor is a non-enhancing astrocytoma or oligodendroglioma. In some embodiments, the brain tumor is a non-enhancing or predominantly non-enhancing astrocytoma or oligodendroglioma. In some embodiments, the brain tumor to be treated is characterized by the presence of an IDH1 mutation, wherein the IDH1 mutation results in accumulation of R(−)-2-hydroxyglutarate in a patient. In one aspect of these embodiments, the IDH1 mutation results in accumulation of R(−)-2-hydroxyglutarate in a patient by providing a new ability of the enzyme to catalyze the NADPH-dependent reduction of α-ketoglutarate to R(−)-2-hydroxyglutarate in a patient. In another aspect of these embodiments, the IDH1 mutation is an R132X mutation. In another aspect of these embodiments, the R132X mutation is selected from R132H, R132C, R132L, R132S and R132G. In another aspect of these embodiments, the R132X mutation is R132H or R132C. In yet another aspect of these embodiments, the R132X mutation is R132H. In still another aspect of these embodiments, at least 30, 40, 50, 60, 70, 80 or 90% of the brain tumor (e.g., glioma) cells carry an IDH1 R132X mutation, such as an R132H, R132C, R132L, R132S or R132G mutation, at the time of diagnosis or treatment. A brain tumor can be analyzed by sequencing cell samples to determine the presence and specific nature of (e.g., the changed amino acid present at) a mutation at amino acid 132 of IDH1.

In other embodiments, the brain tumor to be treated is characterized by the presence of an IDH2 mutation, wherein the IDH2 mutation results in accumulation of R(−)-2-hydroxyglutarate in a patient. In one aspect of these embodiments, the IDH2 mutation results in accumulation of R(−)-2-hydroxyglutarate in a patient by providing a new ability of the enzyme to catalyze the NADPH-dependent reduction of α-ketoglutarate to R(−)-2-hydroxyglutarate in a patient. In another aspect of these embodiments, the mutant IDH2 has an R140X mutation. In another aspect of these embodiments, the R140X mutation is a R140Q mutation. In another aspect of these embodiments, the R140X mutation is a R140W mutation. In another aspect of these embodiments, the R140X mutation is a R140L mutation. In another aspect of these embodiments, the mutant IDH2 has an R172X mutation. In another aspect of these embodiments, the R172X mutation is a R172K mutation. In another aspect of these embodiments, the R172X mutation is a R172G mutation. In still another aspect of these embodiments, at least 30, 40, 50, 60, 70, 80 or 90% of the brain tumor cells carry an IDH2 R140X and/or R172X mutation, such as an R140Q, R140W, or R140L and/or R172K or R172G mutation, at the time of diagnosis or treatment. A brain tumor (e.g., glioma) can be analyzed by sequencing cell samples to determine the presence and specific nature of (e.g., the changed amino acid present at) a mutation at amino acid 140 and/or 172 of IDH2.

In still other embodiments, the brain tumor to be treated is characterized by the presence of an IDH1 mutation and an IDH2 mutation, wherein the IDH1 and IDH2 mutations collectively result in accumulation of R(−)-2-hydroxyglutarate in a patient. In one aspect of these embodiments, the IDH1 and IDH2 mutations result in accumulation of R(−)-2-hydroxyglutarate in a patient by providing a new ability of the enzyme to catalyze the NADPH-dependent reduction of α-ketoglutarate to R(−)-2-hydroxyglutarate in a patient. In various aspects of these embodiments, the IDH1 mutation is an R132X mutation selected from R132H, R132C, R132L, R132V, R132S and R132G. In various aspects of these embodiments, the IDH2 mutation is an R140Q, R140W, R140L, R172K or R172G mutation. In various other aspects of these embodiments, the brain tumor (e.g., glioma) to be treated is characterized by any combination of the foregoing IDH1 and IDH2 mutations. In still other aspects of these embodiments, at least 30, 40, 50, 60, 70, 80 or 90% of the brain tumor cells carry an IDH1 R132X mutation, such as an R132H, R132C, R132L, R132S or R132G mutation, and an IDH2 R140X and/or R172X mutation, such as an R140Q, R140W, or R140L and/or R172K or R172G mutation, at the time of diagnosis or treatment. A brain tumor can be analyzed by sequencing cell samples to determine the presence and specific nature of (e.g., the changed amino acid present at) a mutation at amino acid 132 of IDH1 and at amino acid 140 and/or 172 of IDH2.

In still other embodiments, the brain tumor to be treated is characterized by the presence of an IDH1 allele that does not include an R132X mutation and an IDH2 allele that does not include an R140X or R172X mutation. In one aspect of these embodiments, at least 90% of the brain tumor (e.g., glioma) cells do not include a mutation at amino acid 132 of IDH1 or at amino acid 140 or 172 of IDH2 at the time of diagnosis or treatment. A brain tumor can be analyzed by sequencing cell samples to determine the presence or absence of a mutation at amino acid 132 of IDH1 and at amino acid 140 and/or 172 of IDH2.

The present invention relates to provision of a method for treating a brain tumor with vorasidenib in a patient that has developed elevated liver transaminases upon administration of single or multiple doses of vorasidenib 40 mg QD.

In one embodiment, provided is vorasidenib for use in a method of treating a brain tumor in a patient in a patient that has developed elevated liver transaminases upon administration of single or multiple doses of vorasidenib 40 mg QD.

In one embodiment, provided herein is a pharmaceutical composition comprising 10 mg of vorasidenib or 40 mg of vorasidenib for use in a method for treating a brain tumor, in a patient that has developed elevated liver transaminases upon administration of single or multiple doses of vorasidenib 40 mg QD.

The present invention especially relates to provision of a method of treatment of a brain tumor, characterized by an IDH1 and/or IDH2 mutation, in a patient in need thereof, comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention especially relates to provision of a method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   At 40 mg/day, once a day, if ALT or AST are more than 3-fold but less than or equal to 5 fold of ULN, at first occurrence of the elevation,
   At 20 mg/day, once a day, if ALT or AST are more than 3-fold but less than or equal to 5 fold of ULN, at second occurrence of the elevation,
   At 10 mg/day, once a day, if ALT or AST are more than 3-fold but less than or equal to 5 fold of ULN, at third occurrence of the elevation,
   At 20 mg/day, once a day, if ALT or AST are more than 5-fold but less than or equal to 20 fold of ULN, at first occurrence of the elevation.

The present invention also relates to a method of treatment of a brain tumor, characterized by the presence of a mutant allele of IDH1 and/or IDH2, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubin superior or equal to 2-fold of ULN comprising weekly monitoring Liver Function Tests until resolution.

The present invention relates to vorasidenib for use in a method of treatment of a brain tumor, characterized by an IDH1 and/or IDH2 mutation, in a patient in need thereof, the method comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention relates to vorasidenib for use in a method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   At 40 mg/day, once a day, if ALT or AST are more than 3-fold but less than or equal to 5 fold of ULN, at first occurrence of the elevation,
   At 20 mg/day, once a day, if ALT or AST are more than 3-fold but less than or equal to 5 fold of ULN, at second occurrence of the elevation,
   At 10 mg/day, once a day, if ALT or AST are more than 3-fold but less than or equal to 5 fold of ULN, at third occurrence of the elevation,
   At 20 mg/day, once a day, if ALT or AST are more than 5-fold but less than or equal to 20 fold of ULN, at first occurrence of the elevation.

The present invention also relates to vorasidenib for use in a method of treatment of a brain tumor, characterized by the presence of a mutant allele of IDH1 and/or IDH2, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubin superior or equal to 2-fold of ULN comprising weekly monitoring Liver Function Tests until resolution.

The present invention relates to a pharmaceutical composition comprising 10 mg of vorasidenib or 40 mg of vorasidenib for use in a method of treatment of a brain tumor, characterized by an IDH1 and/or IDH2 mutation, in a patient in need thereof, the method comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention relates to a pharmaceutical composition comprising 10 mg of vorasidenib or 40 mg of vorasidenib for use in a method for treating a brain tumor, characterized by the presence of a mutant allele of IDH1 and/or IDH2, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubin superior or equal to 2-fold of ULN comprising weekly monitoring Liver Function Tests until resolution.

The present invention relates to provision of a method for treating a brain tumor with vorasidenib in a patient with mild or moderate hepatic impairment comprising administering to the patient a dose of 40 mg vorasidenib QD.

In one embodiment, provided is vorasidenib for use in a method of treating a brain tumor in a patient in a patient in a patient with mild or moderate hepatic impairment by administering to the patient 40 mg vorasidenib QD.

In one embodiment, provided is a pharmaceutical composition comprising 10 mg of vorasidenib or 40 mg of vorasidenib for use in a method of treating a brain tumor in a patient in a patient in a patient with mild or moderate hepatic impairment by administering to the patient 40 mg vorasidenib QD.

The present invention especially relates to provision of a method of treatment of a brain tumor, characterized by an IDH1 and/or IDH2 mutation, in a patient with mild or moderate hepatic impairment, comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention relates to vorasidenib for use in a method of treatment of a brain tumor, characterized by an IDH1 and/or IDH2 mutation, in a patient with mild or moderate hepatic impairment comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

The present invention relates to pharmaceutical composition comprising 10 mg of vorasidenib or 40 mg of vorasidenib for use in a method of treatment of a brain tumor, characterized by an IDH1 and/or IDH2 mutation, in a patient with mild or moderate hepatic impairment comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

In some embodiments, the brain tumor is characterized by the presence of an IDH1 mutation, wherein the IDH1 mutation results in accumulation of R(−)-2-hydroxyglutarate in a patient.

In some embodiments, the IDH1 mutation is an R132X mutation.

In some embodiments, the IDH1 mutation is an R132H, R132C, R132S, R132G or R132L mutation.

In some embodiments, the brain tumor is characterized by the presence of an IDH2 mutation, wherein the IDH2 mutation results in accumulation of R(−)-2-hydroxyglutarate in a patient.

In some embodiments, the IDH2 mutation is an R140X mutation.

In some embodiments, the IDH2 mutation is an R140Q, R140W, or R140L mutation.

In some embodiments, the IDH2 mutation is an R172X mutation.

In some embodiments, the IDH2 mutation is an R172K or R172G mutation.

In some embodiments, the brain tumor is characterized by the presence of an IDH1 mutation and an IDH2 mutation, wherein the IDH1 and IDH2 mutations collectively result in accumulation of R(−)-2-hydroxyglutarate in a patient.

In some embodiments, the brain tumor is a glioma. In some embodiments, the brain tumor is oligodendroglioma or astrocytoma.

In some embodiments, the brain tumor is non-enhancing oligodendroglioma or astrocytoma.

In some embodiments, the brain tumor is recurrent or progressive.

In some embodiments, vorasidenib is administered in non-salt form.

In some embodiments, vorasidenib is administered as a cocrystal or a solvate thereof.

In some embodiments, vorasidenib is administered as a cocrystal with citric acid.

In some embodiments, vorasidenib is administered as an hemicitric acid hemihydrate cocrystal.

In some embodiments, vorasidenib is administered at a dose between about 10 mg/day and about 40 mg/day.

In some embodiments, vorasidenib is administered at a dose of about 10 mg/day, about 20 mg/day or about 40 mg/day.

In some embodiments, vorasidenib is administered at a daily dose between about 10 mg, and about 40 mg of vorasidenib.

In some embodiments, vorasidenib is administered at a daily dose of about 10 mg, about 20 mg or about 40 mg of vorasidenib.

In some embodiments, vorasidenib is administered once or twice daily.

In some embodiments, vorasidenib is administered once daily.

The population of patients who may benefit from the methods of treatment of the present invention are patients presenting a hepatic impairment.

The level of hepatic impairment of a patient may be evaluated using the Child-Turcotte-Pugh classification:

| Clinical and lab | Points | | |
|---|---|---|---|
| criteria | 1 | 2 | 3 |
| Encephalopathy | None | Mild to moderate | Severe |
| | | (grade 1 or 2) | (grade 3 or 4) |
| Ascites | None | Mild to moderate | Severe |
| | | (diuretic | (diuretic |
| | | responsive) | refractory) |
| Bilirubin (mg/dL) | <2 | 2-3 | >3 |
| Albumin (g/dL) | >3.5 | 2.8-3.5 | <2.8 |
| Prothrombin time | <4 | 4-6 | >6 |
| Seconds prolonged | <1.7 | 1.7-2.3 | >2.3 |
| International normalized | | | |
| ratio (INR) | | | |

Child-Turcotte-Pugh Class obtained by adding score for each parameter (total points)
Class A = 5 to 6 points (least severe liver disease)
Class B = 7 to 9 points (moderately severe liver disease)
Class C = 10 to 15 points (most severe liver disease)

Level of hepatotoxicity can be classified in grades according to the following criteria:
Grade 1: Alanine aminotransferase (ALT) or Aspartate aminotransferase (AST) increase ≤3× Upper limit of normal (ULN),
Grade 2: ALT or AST >3 but ≤5×ULN,
Grade 3: ALT or AST >5 but ≤20×ULN,
Grade 4: ALT or AST >20×ULN.

### Examples

### Example 1. PKH Study

The influence of hepatic impairment on the PK of vorasidenib was evaluated in a dedicated clinical pharmacology study. Fecal excretion is the predominant route of elimination for vorasidenib (84.7% total radioactivity recovery in feces).

Study PKH-95032-008, a Phase 1, open-label, single-dose study was conducted to evaluate the pharmacokinetics, safety, and tolerability of a 20-mg dose of vorasidenib in subjects with moderate hepatic impairment and matched subjects with normal hepatic function in age, sex, race, BMI and smoking status-matched healthy subjects.

The administration of 20 mg vorasidenib resulted in similar AUC₀₋ₜ and Cₘₐₓ in subjects with normal hepatic function and moderate hepatic impairment.

However, the unbound AUC₀₋ₜ for subjects with moderate hepatic impairment was higher (23%) compared to subjects with normal hepatic function.

Peak exposure was reached at a median Tₘₐₓ of approximately 3 hours and 2 hours in subjects with normal hepatic function and moderate hepatic impairment, respectively.

The unbound plasma protein fraction appeared to be similar in both groups.

Based on the safety and PK data from subjects with moderate hepatic impairment and their healthy matched control, the PK of vorasidenib in subjects with mild hepatic impairment was not investigated further as specified in the protocol.

The summary of PK parameters of vorasidenib is presented in Table 1. Single-dose vorasidenib administration in subjects with moderate hepatic impairment was tolerable.

### Table 1: Summary of Pharmacokinetic Parameters of Vorasidenib 20 mg in Subjects With Normal Hepatic Function and Hepatic Impairment by Child-Pugh Classification

| | Group A | Group B |
|---|---|---|
| Parameter (unit) | (N = 11) | (N = 9) |
| AUC₀₋ₜ (hr · ng/mL) | 1439 (47.5) (11) | 1675 (59.2) (8) |
| AUC₀₋ₜ, u (hr · | 31.95 (47.0) (11) | 48.59(51.5) (8) |
| ng/mL) | | |
| Cₘₐₓ (ng/mL) | 36.55 (41.3) (10) | 38.49 (60.8) (9) |
| Cₘₐₓ, u (ng/mL) | 0.8135 (41.7) (10) | 1.104 (54.2) (9) |
| Tₘₐₓ (h) | 3.000 (0.500, 4.00) (10) | 2.000 (1.00, 3.00) (9) |
| t_{1/2} | 442.6 ± 349.03 (9) | 360.3 ± 189.29 (4) |
| Fu, ave | 2.233 ± 0.24469 (11) | 2.887 ± 0.35364 (n = 9) |

Note:
Group A: Normal Hepatic Function, Group B: Moderate Hepatic Impairment.

The terminal elimination rate constant dependent parameters were excluded from summary statistics if a reliable t1/2 could not be estimated.
Cₘₐₓ and Cₘₐₓ,u for one subject from Group A was excluded from descriptive statistics due to missing sample around Tₘₐₓ (2 h); AUC₀₋ₜ and AUC₀₋ₜ,u for one subject from Group B was excluded from descriptive statistics due to missing last sample (168 h).
AUC₀₋ₜ and Cₘₐₓ are presented as geometric mean (geometric CV %) (n)
Tₘₐₓ is presented as median (minimum, maximum) (n)
All other parameters presented as arithmetic mean±StD

A statistical comparison of total plasma vorasidenib PK parameters in subjects with normal hepatic function and moderate hepatic impairment is presented in Table 2. The GMR of subjects with moderate hepatic impairment to normal hepatic function was 126% for AUC₀₋ₜ and 101% for Cₘₐₓ.

TABLE 2
Statistical Comparison of Total Vorasidenib Plasma Pharmacokinetic
Parameters (Pharmacokinetic Population)

| | | | Ratio (%) of | 90% CI of Ratio of | | |
|---|---|---|---|---|---|---|
| | | | Geometric | Geometric Means | | Inter- |
| | | Geometric | Means (Test/ | (%) | | subject |
| Parameter | Group | Mean | Reference) | Lower | Upper | CV % |
| Cₘₐₓ | Group A | 38.2 | 100.66 | 68.87 | 147.12 | 29.54 |
| (ng/mL) | (N = 11) | [n = 8] | | | | |
| | Group B | 38.5 | | | | |
| | (N = 9) | [n = 9] | | | | |
| AUC₀₋ₜ | Group A | 1390 | 125.97 | 90.01 | 176.28 | 38.86 |
| (hr · ng/mL) | (N = 11) | [n = 9] | | | | |
| | Group B | 1750 | | | | |
| | (N = 9) | [n = 8] | | | | |
| AUC_{inf} | Group A | NE | NE | NE | NE | NE |
| (hr · ng/mL) | (N = 11) | [n = 0] | | | | |
| | Group B | NE | | | | |
| | (N = 9) | [n = 0] | | | | |

Note:
Group A: Normal Hepatic Function, Group B: Moderate Hepatic Impairment (Test).

Geometric means, ratios, and 90% CI are from a linear mixed effects model fitted to the natural log transformed PK parameters with hepatic function group as fixed effect and the matching pair as a random effect.
The ratio and 90% CI of the ratio are presented as a percentage.
AUC₀₋ₜ excluded from inferential statistics due to missing last sample (168 h) for one subject in Group B.
Cₘₐₓ excluded from inferential statistics due to missing sample around Tₘₐₓ (2 h) for one subject in Group A.

### Example 2. INDIGO Study

For Study AG881-C-004, the inclusion criteria specified that subjects with adequate hepatic (serum total bilirubin ≤1.5× upper limit of normal (ULN); and aspartate aminotransferase [AST] or alanine aminotransferase [ALT] at or below ULN; and AP≤2.5×ULN) were eligible for enrollment.

Moderate hepatic impairment (Child-Pugh classification) did not lead to clinically relevant changes in total or free (unbound) vorasidenib exposure following a single oral dose of 20 mg, as seen in the study PKH-95032-008.

The results from a single 20 mg dose can be extrapolated to a single 40 mg dose (the recommended dose) as well as extrapolated to the multiple dose condition of 40 mg QD given that vorasidenib PK is dose proportional from 10 to 200 mg and vorasidenib PK is not time-dependent.

Subjects with moderate and mild hepatic impairment (Child-Pugh classification) can initiate treatment at the proposed recommended dose level for subjects with normal hepatic function.

Although no statistically significant effect of exposure (AUC_{avg} and C_{max,avg}) was identified on ALT, AST, and bilirubin (all grade and Grade ≥2) and the efficacy-response (E-R) analysis indicates the modest changes in exposure will not adversely impact safety/efficacy, monitoring of liver function tests should be performed.

The PK and safety of vorasidenib in patients with severe hepatic impairment (Child-Pugh C) are unknown. For patients with pre-existing severe hepatic impairment, the risks and potential benefits should be considered before initiating treatment with vorasidenib.

In the INDIGO clinical trial (Study AG881-C-004), 18.6% (31/167) of patients treated with Vorasidenib experienced ALT elevations ≥3 times the ULN and 8.4% (14/167) experienced AST elevations >3 times the ULN. Among these patients, 1.2% (2/167) had concurrent ALT or AST elevations >3 times the ULN and total bilirubin ≥2 times the ULN. Liver enzyme and bilirubin increases were transient and improved or resolved with dose modification or permanent discontinuation of treatment. Hepatic failure and hepatic necrosis were observed in one patient treated with vorasidenib and autoimmune hepatitis was observed in one patient treated with vorasidenib.

Monitor liver function tests, including ALT, AST and total bilirubin, prior to the start of Vorasidenib, every 2 weeks during the first 2 months of treatment and then monthly or as clinically indicated. Consider weekly monitoring for ALT or AST elevations ≤3 times the ULN. Withhold, reduce dose or permanently discontinue Vorasidenib based on the severity of the liver function test abnormalities

### Table 3: Recommended Dose Modifications for Hepatotoxicity

| Severity ^{a} | Recommended action | |
|---|---|---|
| ALT or AST increase ≤3 × ULN (Grade 1) | Consider weekly monitoring of LFTs until resolution. | |
| without concurrent total bilirubin >2 × ULN | | |
| ALT or AST >3-20 × ULN (Grade 2 or 3) | Interrupt vorasidenib and monitor LFTs twice per | |
| without concurrent total bilirubin >2 × ULN | week until resolution to Grade 1 or baseline. Restart | |
| | vorasidenib as follows: | |
| | First occurrence: | Resume at 40 mg |
| | ALT or AST >3-5 × | |
| | ULN (Grade 2) | |
| | Second | Resume at 20 mg ^{c} |
| | occurrence: | |
| | ALT or AST >3-5 × | |
| | ULN (Grade 2) | |
| | Third occurrence: | Resume at 10 mg ^{c} |
| | ALT or AST >3-5 × | |
| | ULN (Grade 2) | |
| | First occurrence: | Resume at 20 mg ^{c} |
| | ALT or AST >5- | |
| | 20 × ULN | |
| | (Grade 3) | |
| First occurrence: | Permanently discontinue vorasidenib | |
| Any ALT or AST >20 × ULN (Grade 4), or | and monitor LFTs twice per week until | |
| Any ALT or AST >3-20 × ULN (Grade 2 or | resolution to Grade 1 or baseline. | |
| 3) with concurrent total bilirubin >2 × ULN in | | |
| absence of clear alternative explanation ^{b} | | |
| Second occurrence: | | |
| ALT or AST >5 × ULN regardless of cause | | |
| Fourth occurrence: | | |
| ALT or AST >3-5 × ULN (Grade 2) | | |
| regardless of cause | | |

Abbreviations:
ALT = Alanine aminotransferase;
AST = Aspartate aminotransferase;
LFT = Liver function test;
ULN = Upper limit of normal
^{a} Adverse reactions graded by the National Cancer Institute Common Terminology Criteria for Adverse Events (NCI-CTCAE). Grade 1 is mild, Grade 2 is moderate, Grade 3 is severe and Grade 4 is life-threatening.
^{b} If an alternative aetiology is identified, consider resuming vorasidenib at 20 mg following resolution to Grade 1 or baseline.
^{c} Dose re-escalation may be considered after 3 months if no LFT elevations are observed. Manage recurrence of LFT elevations after dose reduction by further dose reduction or discontinuation.

In Study AG881-C-004, among subjects in the vorasidenib 40 mg QD arm (N=167) and in the placebo arm (N=163), a higher incidence of subjects with TEAEs occurred in the vorasidenib arm (73 [43.7%] subjects vs. 34 [20.9%] subjects, respectively) (Table 4). Similarly, more subjects in the vorasidenib arm experienced treatment-related TEAEs than the placebo arm (65 [38.9%] vs. 26 [16.0%] subjects, respectively). More subjects in the vorasidenib arm had TEAEs that led to study treatment modifications including treatment discontinuation (5 [3.0%] subjects), interruption (28 [16.8%] subjects) and dose reduction (14 [8.4%] subjects) compared with subjects in the placebo arm (0, 5 [3.1%], and 2 [1.2%] subjects, respectively). A summary of the liver enzyme elevation events is shown in Table 4.

Table 4. Overall Summary of Treatment-Emergent Adverse Events from SMQ (Broad Search) of Hepatic Enzymes Elevation Liver-Related Investigations, Signs and Symptoms—Glioma with Vorasidenib 40 mg QD

| | Vorasidenib 40 mg QD ^{a} | | | | Placebo | |
|---|---|---|---|---|---|---|
| | AG881-C-004 | | AG881-C-004 | | AG881-C-004 | |
| | without crossover ^{b} | | post-crossover ^{c} | | pre-crossover ^{e} | |
| Number (%) of subjects | N = 167 | | N = 52 | | N = 163 | |
| with | n (%) | | n (%) | | n (%) | |
| Any TEAEs | 73 | (43.7) | 15 | (28.8) | 34 | (20.9) |
| Grade ≥2 TEAEs | 35 | (21.0) | 7 | (13.5) | 10 | (6.1) |
| Grade ≥3 TEAEs | 19 | (11.4) | 3 | (5.8) | 2 | (1.2) |
| Treatment-related | 65 | (38.9) | 10 | (19.2) | 26 | (16.0) |
| TEAEs | | | | | | |
| Grade ≥2 Treatment- | 34 | (20.4) | 5 | (9.6) | 6 | (3.7) |
| related TEAEs | | | | | | |
| Grade ≥3 Treatment- | 18 | (10.8) | 3 | (5.8) | 1 | (0.6) |
| related TEAEs | | | | | | |
| Serious TEAEs | 1 | (0.6) | 1 | (1.9) | 0 | |
| Serious treatment-related | 1 | (0.6) | 1 | (1.9) | 0 | |
| TEAEs | | | | | | |
| TEAEs leading to study | 5 | (3.0) | 1 | (1.9) | 0 | |
| treatment | | | | | | |
| discontinuation | | | | | | |
| TEAEs leading to study | 28 | (16.8) | 7 | (13.5) | 5 | (3.1) |
| treatment interruption | | | | | | |
| TEAEs leading to study | 14 | (8.4) | 2 | (3.8) | 2 | (1.2) |

| | | | |
|---|---|---|---|
| treatment dose reduction | | | |
| TEAEs leading to death | 0 | 0 | 0 |
| Treatment-related TEAEs | 0 | 0 | 0 |
| leading to death | | | |

Most of the TEAEs were managed within the clinical trial protocol, and discontinuations due to elevated liver enzyme effects were rare (5 subjects, 3%). The median time to resolution (TTR) for the most common (occurred in ≥2 subjects) Grade ≥3 TEAEs in subjects in the vorasidenib arm are as follows:

The median TTR for alanine aminotransferase increased (any grade) was longer for subjects in the vorasidenib arm than in the placebo arm (56.0 days [range: 5-389 days] vs. 28.5 days [range: 4-113 days]). The number of subjects who experienced a TTR in ≤15 days or >60 days differed between the vorasidenib and placebo arms (≤15 days: 8 [9.3%] vs. 11 [30.6%], respectively; >60 days: 36 [41.9%] vs. 4 [11.1%], respectively).

The median TTR for aspartate aminotransferase increased (any grade), was the same in both arms at 29.0 days (range: 5-537 days for the vorasidenib arm and 6-162 days for placebo). The number of subjects experiencing a TTR in ≤15 days was 20 (28.6%) in the vorasidenib arm and 7 (38.9%) in placebo, while 15 (21.4%) subjects in the vorasidenib arm had a TTR in >60 days vs. 1 (5.6%) in placebo.

### Exposure-Efficacy Analysis

E-R analyses for efficacy were conducted using data from subjects with glioma from Study AG881-C-004 only. The exposure-efficacy analysis dataset was comprised of 167 subjects with glioma, including the 5 subjects who started treatment with 50 mg QD Formulation 1 at C1D1, but then switched to 40 mg QD Formulation 2 during the treatment. Exposure-efficacy analyses included clinical response to treatment, i.e., PFS, TTNI, OR (CR+PR+mR), CR+PR, all defined per blind independent review committee (BIRC). The number of subjects with OR, CR+PR, progression (PFS) and intervention (TTNI) was 18 (10.8%, only PR and MR), 2 (1.2%, only PR), 47 (28.1%), and 19 (11.4%), respectively.

The exposure metrics were pop PK model predicted individual exposures by using post-hoc individual PK parameters and were based on the actual average dose across the treatment duration. The dose of vorasidenib used in the simulations is corrected for dose-reductions and interruptions occurring during the treatment in real life. The actual average dose for subjects treated with 40 mg QD Formulation 2 formulation in Study AG881-C-004 was 37.8 mg QD.

The exposure-safety analysis used AUC_{avg} and C_{maxavg} at steady state based on the actual average dose up to the time of an event, up to the end of treatment (EOT) or up to the study data cut-off in case a subject did not experience a TEAE. The exposure-efficacy analysis used the area under the curve (AUCₑₒₜ) at steady state based on the actual average dose across the treatment duration until EOT.

Over the 10 to 300 mg QD dose range (Formulation 1), the geometric mean steady-state plasma exposure for AUC₀₋ₜₐᵤ ranged from 462 to 8604 h*ng/mL and the geometric mean Cₘₐₓ ranged from 38.9 to 526 ng/mL at C1D15. At the therapeutic dose of 40 mg QD (Formulation 2), the PopPK model predicted geometric mean AUC,ₛₛ and C_{max,ss} were 3 times lower than corresponding exposure metrics at 300 mg QD (Formulation 1). For most of the safety endpoints, a flat E-R relationship was observed with no clear trend of incidence of events across exposure quartiles. The proportion of subjects with event occurrence for both PFS and TTNI or with response for both OR (ie, CR, partial response [PR], or mR) and CR+PR was similar across exposure quartiles and suggested that no E-R relationship was observed in Study AG881-C-004.

Exposure did not affect efficacy responses in the studied exposure range. Flat E-R profiles were observed for PFS, TTNI, OR and CR+PR, supporting that the efficacy benefit was consistent across the range of exposures achieved in Study AG881-C-004.

Overall, in Study AG881-C-004, 27 (16.2%) subjects in the vorasidenib arm had at least 1 dose reduction based on the exposure data. Of note, these data include any reduced dose irrespective of reason, including subject error. Specifically, TEAEs leading to dose reduction were reported in 18 (10.8%) subjects in the vorasidenib arm of which the most common TEAE was alanine aminotransferase increased (N=13 [7.8%]). For the 14 subjects with dose reductions due to hepatotoxicity AE, a swimming plot showing time on study by individual subject is provided in the Figure. The plot includes details regarding timing and duration of TEAEs leading to dose reduction, timing and duration of reduced dose, as well as disease response by Blinded Independent Central Review (BIRC) at each assessment timepoint.

Of the 14 subjects who experienced a TEAE within the hepatotoxicity SMQ search strategy (hereafter referred to as TEAE for simplicity) leading to dose reduction, 10 subjects required a dose reduction from 40 mg QD to 20 mg QD only, and 4 subjects required a second dose reduction from 20 mg QD to 10 mg QD.

Across the 10 subjects who had a dose reduction from 40 mg QD to 20 mg QD only, the timing of the dose reductions (first occurrence of 20 mg QD if subjects had multiple) ranged from Study Day 72 to Study Day 202. As of the data cutoff (6 Sep. 2022), 7 subjects remained on treatment with 20 mg QD; 2 subjects discontinued treatment due to centrally confirmed radiographic PD (840110-102, 840176-106), and 1 due to AE (826113-103, remained on study in PFS follow up with continued disease response assessments). Across the 10 subjects who had a dose reduction from 40 mg QD to 20 mg QD only, 8 subjects had a best response of SD, and 1 subject had a best response of MR, and one had best response of PD by BIRC at the closest response assessment while receiving 20 mg QD. Two of the subjects, 840168-105 and 840176-106, remained on treatment beyond an assessment of PD by BIRC. Per protocol, central confirmation of disease progression by BIRC prior to unblinding of treatment assignment only occurs following an assessment of PD by the Investigator. For these subjects the disease response was assessed as SD by the Investigator at these timepoints; therefore, central confirmation of PD was not performed and subjects remained on study treatment.

Across the 4 subjects who had a further reduction from 20 mg QD to 10 mg QD, the timing of the start of dose reductions to 10 mg due to TEAEs ranged from Study Day 114 to Study Day 297. As of the data cutoff date, 3 of the 4 subjects who had dose reductions to 10 mg due to a TEAE remain on treatment at 10 mg QD, and 1 subject (840110-104) dose escalated to 20 mg and remains on treatment at 20 mg QD. Three of the 4 subjects who had dose reductions to 10 mg due to a TEAE had a best response of SD, and 1 subject had a response of PD while receiving 10 mg QD. Two subjects, 840182-106 and 840110-104, continued treatment after PD as assessed by BIRC; however, in both cases, the Investigator assessment at these timepoints was SD, therefore central confirmation of PD was not performed, and the subjects remained on study treatment.

These data support the dose reduction guidelines included in Table 3.

### Example 3—Results from a Phase 1, Multicenter, Randomized, Open-Label, Perioperative Study of Vorasidenib (AG-881) in Patients with Recurrent, Nonenhancing, IDH1-Mutant, Low-Grade Glioma—ClinicalTrials.Gov NCT03343197

The primary objective of this perioperative study was to determine the 2-HG concentration in tumors resected following presurgical treatment with vorasidenib (AG-881) compared with untreated control tumors in patients with recurrent, nonenhancing, mIDH1-R132H LGG.

Briefly, patients with recurrent non-enhancing WHO-2016 Grade (Gr) 2 or 3 mIDH1-R132H oligodendroglioma or astrocytoma undergoing craniotomy were randomized to receive vorasidenib 10 mg QD and 50 mg QD, or no treatment for 4 wks preoperatively in Cohort 1. Tumors were assessed for mIDH1 status, cellularity, 2-HG, and drug concentration. Treated samples were compared to control patients and mIDH1 and wild type (WT) banked reference (ref) samples. Plasma and CSF 2-HG were assessed. Patients with non-evaluable tissue were replaced.

The study was conducted with an early formulation of vorasidenib hemicitric acid hemihydrate co-crystal (F1) shown in Table 5.

In this study, vorasidenib concentration (mean±StD) in brain tumor tissue collected after approximately 28 days of dosing was 75.8+23.0 ng/g in subjects who received 10 mg QD (F1) and 118.5+49.5 ng/g in subjects who received 50 mg QD (F1).

The study further identified the posterior median percentage reduction in tumor 2-HG levels of 63.5% (with a 95% CI of 22.2% to 88.4%) for subjects treated with 10 mg QD (F1) and 92.6% (with a 95% CI of 76.1% to 97.6%) for subjects treated with 50 mg QD (F1) in comparison to untreated subjects.

### Example 4—Vorasidenib Formulations

Two exemplary vorasidenib formulations—F1 (used in early clinical development, including the study described in Example 3) and F2 (used in the study described in Example 2) the are shown in Table 5

| | | | | Formulation F2 |
|---|---|---|---|---|
| | | Formulation F1 | | (% compo- |
| | | (% composition) | | sition) |
| | | | 25 mg and | 10 mg, |
| Component | Function | 5 mg | 100 mg | 40 mg, 50 mg |

Core tablets

| | | | | |
|---|---|---|---|---|
| Vorasidenib | Active | 6.27 | 31.3 | 12.2 |
| citrate | | | | |
| Microcrystalline | Diluent | 83.7 | 54.2 | 57.3 |
| cellulose | | | | |
| Croscarmellose | Disintegrant | 6.00 | 8.00 | 4.85 |
| sodium | | | | |
| Hydroxypropyl | Binder | 2.00 | 2.00 | |
| cellulose | | | | |
| Colloidal silicon | Glidant | | 2.00 | |
| dioxide | | | | |
| Silicified | Diluent/ | | | 20.4 |
| microcrystalline | glidant | | | |
| cellulose | | | | |
| Sodium lauryl | Wetting | 1.00 | 1.00 | 0.970 |
| sulfate | agent | | | |
| Magnesium | Lubricant | 1.00 | 1.50 | 1.46 |
| stearate | | | | |

Film Coating

| | | |
|---|---|---|
| Opadry ® II | Coating agent | 2.91 |
| white | | |
| Purified Water | Coating solvent | |

Total

| | | |
|---|---|---|
| 100 | 100 | 100 |

The bioavailability of F2 has been determined to be 50% higher than that of F1. Therefore, it is anticipated that the 10 mg and 20 mg F2 doses will achieve brain tumor concentrations that are within the range of what was observed with the 10 mg and 50 mg F1 doses. To further confirm this hypothesis, PK/PD modeling was undertaken by fitting 2-HG concentration in brain tumors vs. plasma vorasidenib data before surgery. The model predicts that the median suppression levels of 2-HG for daily doses of 10 mg and 20 mg F2 are 71.2% and 81.2%, respectively, within the range of 2-HG suppression following the 10 mg and 50 mg F1. According to these predictions, both doses achieve 2-HG suppression corresponding to greater than 10 mg F1, thereby demonstrating target engagement.

With the improved bioavailability of F2 compared with F1, vorasidenib's predicted inhibition of 2-HG at the target site was within the range observed for the 10 mg to 50 mg F1 doses, where dose-dependent target engagement was demonstrated. Thus, a dose reduction from 40 mg QD to 20 mg or 10 mg QD due to tolerability is justified from a pharmacokinetic standpoint.

### ENUMERATED EMBODIMENTS

Embodiment 1. Method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   a. At 40 mg/day, once a day, if ALT or AST are more than 3-fold but less than or equal to 5 fold of ULN, at first occurrence of the elevation,
   b. At 20 mg/day, once a day, if ALT or AST are more than 3-fold but less than or equal to 5 fold of ULN, at second occurrence of the elevation,
   c. At 10 mg/day, once a day, if ALT or AST are more than 3-fold but less than or equal to 5 fold of ULN, at third occurrence of the elevation,
   d. At 20 mg/day, once a day, if ALT or AST are more than 5-fold but less than or equal to 20 fold of ULN, at first occurrence of the elevation.

Embodiment 2. Method of treatment of a brain tumor, characterized by an IDH1 and/or IDH2 mutation, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubon superior or equal to 2-fold of ULN comprising weekly monitoring Liver Function Tests until resolution.

Embodiment 3. Method for treating a brain tumor with vorasidenib in a patient with mild or moderate hepatic impairment comprising administering to the patient a dose of 40 mg vorasidenib QD.

Embodiment 4. Method of treatment of a brain tumor, characterized by an IDH1 and/or IDH2 mutation, in a patient with mild or moderate hepatic impairment, comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
   III. At 10 mg/day, once a day, if ALT or AST are more than 3-fold but less than or equal to 5 fold of ULN, at third occurrence of the elevation,
   IV. At 20 mg/day, once a day, if ALT or AST are more than 5-fold but less than or equal to 20 fold of ULN, at first occurrence of the elevation.

Embodiment 5. Method according to any one of embodiments 1 to 4, characterized in that the brain tumor is a glioma.

Embodiment 6. Method according to any one of embodiments 1 to 5, characterized in that the brain tumor is oligodendroglioma.

Embodiment 7. Method according to any one of embodiments 1 to 5, characterized in that the brain tumor is astrocytoma.

Embodiment 8. Method according to any one of embodiments 1 to 7, characterized in that the brain tumor is recurrent or progressive.

Embodiment 9. Method according to any one of embodiments 1 to 5 or 8, characterized in that the brain tumor is a predominantly non-enhancing astrocytoma or oligodendroglioma.

Embodiment 10. Method according to any one of embodiments 1 to 9, characterized in that the brain tumor presents an IDH1 mutation.

Embodiment 11. Method according to embodiment 10, characterized in that the presence of IDH1 mutation results in accumulation of R(−)-2-hydroxyglutarate in a patient.

Embodiment 12. Method according to embodiment 10 or 11, characterized in that the IDH1 mutation is a R132X mutation.

Embodiment 13. Method according to embodiment 12, characterized in that the IDH1 mutation is a R132H, R132C, R132S, R132G or R132L mutation.

Embodiment 14. Method according to any one of embodiments 1 to 13, characterized in that the brain tumor presents an IDH2 mutation.

Embodiment 15. Method according to embodiment 14, characterized in that the presence of IDH2 mutation results in accumulation of R(−)-2-hydroxyglutarate in a patient.

Embodiment 16. Method according to embodiment 14 or 15, characterized in that the IDH2 mutation is a R140X mutation.

Embodiment 17. Method according to embodiment 16, characterized in that the IDH2 mutation is a R140Q, R140W, or R140L mutation.

Embodiment 18. Method according to embodiment 14 or 15, characterized in that the IDH2 mutation is a R172X mutation.

Embodiment 19. Method according to embodiment 18, characterized in that the IDH2 mutation is a R172K or R172G mutation.

Embodiment 20. Method according to any one of embodiments 1 to 19, characterized in that the brain tumor presents an IDH1 and an IDH2 mutation.

Embodiment 21. Method according to embodiment 20, characterized in that the IDH1 and the IDH2 mutation result collectively in accumulation of R(−)-2-hydroxyglutarate in a patient.

Embodiment 22. Method according to any one of embodiments 1 to 21, characterized in that vorasidenib is administered as a cocrystal with citric acid.

Embodiment 23. Method according to embodiment 22, characterized in that vorasidenib is administered as a hemicitric acid hemihydrate cocrystal.

Embodiment 24. Vorasidenib for use in a method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   a. At 40 mg/day, once a day, if ALT or AST are more than 3-fold but less than or equal to 5 fold of ULN, at first occurrence of the elevation,
   b. At 20 mg/day, once a day, if ALT or AST are more than 3-fold but less than or equal to 5 fold of ULN, at second occurrence of the elevation,
   c. At 10 mg/day, once a day, if ALT or AST are more than 3-fold but less than or equal to 5 fold of ULN, at third occurrence of the elevation,
   d. At 20 mg/day, once a day, if ALT or AST are more than 5-fold but less than or equal to 20 fold of ULN, at first occurrence of the elevation.

Embodiment 25. Vorasidenib for use in a method of treatment of a brain tumor, characterized by an IDH1 and/or IDH2 mutation, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubon superior or equal to 2-fold of ULN comprising weekly monitoring Liver Function Tests until resolution.

Embodiment 26. Vorasidenib for use in a method of treating a brain tumor in a patient in a patient in a patient with mild or moderate hepatic impairment by administering to the patient 40 mg vorasidenib QD.

Embodiment 27. Vorasidenib for use in a method of treatment of a brain tumor, characterized by an IDH1 and/or IDH2 mutation, in a patient with mild or moderate hepatic impairment comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation.

Embodiment 28. Vorasidenib for use according to any one of embodiments 24 to 27, characterized in that the brain tumor is a glioma.

Embodiment 29. Vorasidenib for use according to any one of embodiments 24 to 28, characterized in that the brain tumor is oligodendroglioma.

Embodiment 30. Vorasidenib for use according to any one of embodiments 24 to 28, characterized in that the brain tumor is astrocytoma.

Embodiment 31. Vorasidenib for use according to any one of embodiments 24 to 30, characterized in that the brain tumor is recurrent or progressive.

Embodiment 32. Vorasidenib for use according to any one of embodiments 24 to 28 or 31, characterized in that the brain tumor is a predominantly non-enhancing astrocytoma or oligodendroglioma.

Embodiment 33. Vorasidenib for use according to any one of embodiments 24 to 32, characterized in that the brain tumor presents an IDH1 mutation.

Embodiment 34. Vorasidenib for use according to embodiment 33, characterized in that the presence of IDH1 mutation results in accumulation of R(−)-2-hydroxyglutarate in a patient.

Embodiment 35. Vorasidenib for use according to embodiment 33 or 34, characterized in that the IDH1 mutation is a R132X mutation.

Embodiment 36. Vorasidenib for use according to embodiment 35, characterized in that the IDH1 mutation is a R132H, R132C, R132S, R132G or R132L mutation.

Embodiment 37. Vorasidenib for use according to any one of embodiments 24 to 36, characterized in that the brain tumor presents an IDH2 mutation.

Embodiment 38. Vorasidenib for use according to embodiment 37, characterized in that the presence of IDH2 mutation results in accumulation of R(−)-2-hydroxyglutarate in a patient.

Embodiment 39. Vorasidenib for use according to embodiment 37 or 38, characterized in that the IDH2 mutation is a R140X mutation.

Embodiment 40. Vorasidenib for use according to embodiment 39, characterized in that the IDH2 mutation is a R140Q, R140W, or R140L mutation.

Embodiment 41. Vorasidenib for use according to embodiment 37 or 38, characterized in that the IDH2 mutation is a R172X mutation.

Embodiment 42. Vorasidenib for use according to embodiment 41, characterized in that the IDH2 mutation is a R172K or R172G mutation.

Embodiment 43. Vorasidenib for use according to any one of embodiments 24 to 42, characterized in that the brain tumor presents an IDH1 and an IDH2 mutation.

Embodiment 44. Vorasidenib for use according to embodiment 43, characterized in that the IDH1 and the IDH2 mutation result collectively in accumulation of R(−)-2-hydroxyglutarate in a patient.

Embodiment 45. Vorasidenib for use according to any one of embodiments 24 to 44, characterized in that vorasidenib is administered as a cocrystal with citric acid.

Embodiment 46. Vorasidenib for use according to embodiment 45, characterized in that vorasidenib is administered as a hemicitric acid hemihydrate cocrystal.

Embodiment 47. Pharmaceutical composition comprising 10 mg of vorasidenib or 40 mg of vorasidenib for use in a method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
   a. At 40 mg/day, once a day, if ALT or AST are more than 3-fold but less than or equal to 5 fold of ULN, at first occurrence of the elevation,
   b. At 20 mg/day, once a day, if ALT or AST are more than 3-fold but less than or equal to 5 fold of ULN, at second occurrence of the elevation,
   c. At 10 mg/day, once a day, if ALT or AST are more than 3-fold but less than or equal to 5 fold of ULN, at third occurrence of the elevation,
   d. At 20 mg/day, once a day, if ALT or AST are more than 5-fold but less than or equal to 20 fold of ULN, at first occurrence of the elevation.

Embodiment 48. Pharmaceutical composition comprising 10 mg of vorasidenib or 40 mg of vorasidenib for use in a method of treatment of a brain tumor, characterized by an IDH1 and/or IDH2 mutation, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubon superior or equal to 2-fold of ULN comprising weekly monitoring Liver Function Tests until resolution.

Embodiment 49. Pharmaceutical composition comprising 10 mg of vorasidenib or 40 mg of vorasidenib for use in a method of treating a brain tumor in a patient in a patient in a patient with mild or moderate hepatic impairment by administering to the patient 40 mg vorasidenib QD.

Embodiment 50. Pharmaceutical composition comprising 10 mg of vorasidenib or 40 mg of vorasidenib for use in a method of treatment of a brain tumor, characterized by an IDH1 and/or IDH2 mutation, in a patient with mild or moderate hepatic impairment comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
   (i) monitoring the Liver Function Test (LFT) twice per week until resolution to grade I or baseline;
   (ii) restarting vorasidenib:
      I. At 40 mg/day, once a day, if ALT or AST are more than 3-fold but less than or equal to 5 fold of ULN, at first occurrence of the elevation,
      II. At 20 mg/day, once a day, if ALT or AST are more than 3-fold but less than or equal to 5 fold of ULN, at second occurrence of the elevation,
      III. At 10 mg/day, once a day, if ALT or AST are more than 3-fold but less than or equal to 5 fold of ULN, at third occurrence of the elevation,
      IV. At 20 mg/day, once a day, if ALT or AST are more than 5-fold but less than or equal to 20 fold of ULN, at first occurrence of the elevation

Embodiment 51. Pharmaceutical composition comprising 10 mg of vorasidenib or 40 mg of vorasidenib for use according to any one of embodiments 47 to 50, characterized in that the brain tumor is a glioma.

Embodiment 52. Pharmaceutical composition comprising 10 mg of vorasidenib or 40 mg of vorasidenib for use according to any one of embodiments 47 to 51, characterized in that the brain tumor is oligodendroglioma.

Embodiment 53. Pharmaceutical composition comprising 10 mg of vorasidenib or 40 mg of vorasidenib for use according to any one of embodiments 47 to 51, characterized in that the brain tumor is astrocytoma.

Embodiment 54. Pharmaceutical composition comprising 10 mg of vorasidenib or 40 mg of vorasidenib for use according to any one of embodiments 47 to 53, characterized in that the brain tumor is recurrent or progressive.

Embodiment 55. Pharmaceutical composition comprising 10 mg of vorasidenib or 40 mg of vorasidenib for use according to any one of embodiments 47 to 51 or 54, characterized in that the brain tumor is a predominantly non-enhancing astrocytoma or oligodendroglioma.

Embodiment 56. Pharmaceutical composition comprising 10 mg of vorasidenib or 40 mg of vorasidenib for use according to any one of embodiments 47 to 55, characterized in that the brain tumor presents an IDH1 mutation.

Embodiment 57. Pharmaceutical composition comprising 10 mg of vorasidenib or 40 mg of vorasidenib for use according to embodiment 56, characterized in that the presence of IDH1 mutation results in accumulation of R(−)-2-hydroxyglutarate in a patient.

Embodiment 58. Pharmaceutical composition comprising 10 mg of vorasidenib or 40 mg of vorasidenib for use according to embodiment 56 or 57, characterized in that the IDH1 mutation is a R132X mutation.

Embodiment 59. Pharmaceutical composition comprising 10 mg of vorasidenib or 40 mg of vorasidenib for use according to embodiment 58, characterized in that the IDH1 mutation is a R132H, R132C, R132S, R132G or R132L mutation.

Embodiment 60. Pharmaceutical composition comprising 10 mg of vorasidenib or 40 mg of vorasidenib for use according to any one of embodiments 47 to 59, characterized in that the brain tumor presents an IDH2 mutation.

Embodiment 61. Pharmaceutical composition comprising 10 mg of vorasidenib or 40 mg of vorasidenib for use according to embodiment 60, characterized in that the presence of IDH2 mutation results in accumulation of R(−)-2-hydroxyglutarate in a patient.

Embodiment 62. Pharmaceutical composition comprising 10 mg of vorasidenib or 40 mg of vorasidenib for use according to embodiment 60 or 61, characterized in that the IDH2 mutation is a R140X mutation.

Embodiment 63. Pharmaceutical composition comprising 10 mg of vorasidenib or 40 mg of vorasidenib for use according to embodiment 62, characterized in that the IDH2 mutation is a R140Q, R140W, or R140L mutation.

Embodiment 64. Pharmaceutical composition comprising 10 mg of vorasidenib or 40 mg of vorasidenib for use according to embodiment 60 or 61, characterized in that the IDH2 mutation is a R172X mutation.

1. Method of treatment of a brain tumor, characterized by an IDH1 and/or IDH2 mutation, in a patient treated by vorasidenib 40 mg/day, once a day, and presenting an elevation of more than 3-fold but less than or equal to 20 fold of the upper limit of normal (ULN) of Alanine aminotransferase (ALT) or Aspartate aminotransferase (AST) comprising:
interrupting the treatment with vorasidenib,
monitoring the Liver Function Test (LFT) twice per week until resolution to grade I or baseline,
restarting vorasidenib:
a. At 40 mg/day, once a day, if ALT or AST are more than 3-fold but less than or equal to 5 fold of ULN, at first occurrence of the elevation,
b. At 20 mg/day, once a day, if ALT or AST are more than 3-fold but less than or equal to 5 fold of ULN, at second occurrence of the elevation,
c. At 10 mg/day, once a day, if ALT or AST are more than 3-fold but less than or equal to 5 fold of ULN, at third occurrence of the elevation,
d. At 20 mg/day, once a day, if ALT or AST are more than 5-fold but less than or equal to 20 fold of ULN, at first occurrence of the elevation.

2. Method of treatment of a brain tumor, characterized by an IDH1 and/or IDH2 mutation, in a patient treated by vorasidenib 40 mg/day, once a day, and presenting an elevation inferior or equal to 3-fold of the upper limit of normal (ULN) of Alanine aminotransferase (ALT) or Aspartate aminotransferase (AST) without concurrent elevation of total bilirubon superior or equal to 2-fold of ULN comprising weekly monitoring Liver Function Tests until resolution.

3. Method for treating a brain tumor with vorasidenib in a patient with mild or moderate hepatic impairment comprising administering to the patient a dose of 40 mg vorasidenib QD.

4. Method of treatment of a brain tumor, characterized by an IDH1 and/or IDH2 mutation, in a patient with mild or moderate hepatic impairment, comprising
(a) administering to the patient vorasidenib 40 mg/day, once a day (QD);
(b) monitoring elevation of liver enzymes;
(c) if an elevation inferior or equal to 3-fold of the upper limit of normal (ULN) of Alanine aminotransferase (ALT) or Aspartate aminotransferase (AST) occurs without concurrent elevation of total bilirubin superior or equal to 2-fold of ULN then monitoring Liver Function Tests weekly until resolution;
(d) if an elevation of more than 3-fold but less than or equal to 20 fold of the upper limit of normal (ULN) of Alanine aminotransferase (ALT) occurs, then;
(i) monitoring the Liver Function Test (LFT) twice per week until resolution to grade I or baseline;
(ii) restarting vorasidenib:
I. At 40 mg/day, once a day, if ALT or AST are more than 3-fold but less than or equal to 5 fold of ULN, at first occurrence of the elevation,
II. At 20 mg/day, once a day, if ALT or AST are more than 3-fold but less than or equal to 5 fold of ULN, at second occurrence of the elevation,
III. At 10 mg/day, once a day, if ALT or AST are more than 3-fold but less than or equal to 5 fold of ULN, at third occurrence of the elevation,
IV. At 20 mg/day, once a day, if ALT or AST are more than 5-fold but less than or equal to 20 fold of ULN, at first occurrence of the elevation.

5. Method according to any one of claims 1 to 4, characterized in that the brain tumor is a glioma.

6. Method according to any one of claims 1 to 5, characterized in that the brain tumor is oligodendroglioma.

7. Method according to any one of claims 1 to 5, characterized in that the brain tumor is astrocytoma.

8. Method according to any one of claims 1 to 7, characterized in that the brain tumor is recurrent or progressive.

9. Method according to any one of claims 1 to 5 or 8, characterized in that the brain tumor is a predominantly non-enhancing astrocytoma or oligodendroglioma.

10. Method according to any one of claims 1 to 9, characterized in that the brain tumor presents an IDH1 mutation.

11. Method according to claim 10, characterized in that the presence of IDH1 mutation results in accumulation of R(−)-2-hydroxyglutarate in a patient.

12. Method according to claim 10 or 11, characterized in that the IDH1 mutation is a R132X mutation.

13. Method according to claim 12, characterized in that the IDH1 mutation is a R132H, R132C, R132S, R132G or R132L mutation.

14. Method according to any one of claims 1 to 13, characterized in that the brain tumor presents an IDH2 mutation.

15. Method according to claim 14, characterized in that the presence of IDH2 mutation results in accumulation of R(−)-2-hydroxyglutarate in a patient.

16. Method according to claim 14 or 15, characterized in that the IDH2 mutation is a R140X mutation.

17. Method according to claim 16, characterized in that the IDH2 mutation is a R140Q, R140W, or R140L mutation.

18. Method according to claim 14 or 15, characterized in that the IDH2 mutation is a R172X mutation.

19. Method according to claim 18, characterized in that the IDH2 mutation is a R172K or R172G mutation.

20. Method according to any one of claims 1 to 19, characterized in that the brain tumor presents an IDH1 and an IDH2 mutation.

21. Method according to claim 20, characterized in that the IDH1 and the IDH2 mutation result collectively in accumulation of R(−)-2-hydroxyglutarate in a patient.

22. Method according to any one of claims 1 to 21, characterized in that vorasidenib is administered as a cocrystal with citric acid.

23. Method according to claim 22, characterized in that vorasidenib is administered as a hemicitric acid hemihydrate cocrystal.
